(19) United States
(12) Patent Application Publication
(10) Pub. No.: US 2025/0000509 A1
(43) Pub. Date: Jan. 2, 2025

(54) STAPLING DEVICE INCLUDING A RETRACTABLE KNIFE ASSEMBLY

(71) Applicant: Covidien LP, Mansfield, MA (US)

(72) Inventor: Zhaokai Wang, Shanghai (CN)

(21) Appl. No.: 18/707,460

(22) PCT Filed: Nov. 12, 2021
(86) PCT No.: PCT/CN2021/130302

§ 371 Date: May 3, 2024

(51) Int. Cl. A61B 17/072

(52) U.S. Cl. CPC A61B 17/072; A61B 2017/07257; A61B 2017/07271; A61B 2017/07285

(57) ABSTRACT

A stapling device (10) includes a handle assembly (12), an elongate body (14), a clamp slide member (66), and an end effector (16). The handle assembly (12) includes a movable trigger (22) and a cam member (700) operatively coupled to the movable trigger (22). The cam member (700) includes lateral walls (700a, 700b) that flexibly oppose each other and define a gap (710) between the lateral walls (700a, 700b). The lateral walls (700a, 700b) include respective camming protrusions (702a, 702b) extending towards each other. The clamp slide member (66) has a distal portion (68) that defines a curved pocket (77) and a proximal portion (69) that defines a cam slot (84) configured to slidably receive the camming protrusions (702a, 702b) of the cam member (700) such that actuation of the movable trigger (22) causes axial displacement of the clamp slide member (66). The end effector (16) is supported on the distal portion of the elongate body (14). The end effector (16) includes an end effector frame (56), an anvil assembly (11), and a cartridge assembly (50).

### FIELD

The disclosure is generally related to surgical stapling devices and, more particularly, to surgical stapling devices including a retractable knife blade assembly.

### BACKGROUND

Surgical stapling devices for applying rows of staples through compressed living tissue are known in the art and are commonly used for closure of tissue or organs prior to transection or resection of the tissue and for occlusion of the organs during thoracic or abdominal procedures. Such stapling devices include a knife for transecting or resecting the tissue or organs simultaneously with application of the staples to the tissue.

Typically, the stapling devices include a tool assembly having an anvil assembly and a cartridge assembly, an approximation assembly for moving the tool assembly between spaced and clamped positions, and a drive member for advancing a pusher to eject staples from the cartridge assembly. The tool assembly is available in a variety of configurations including linear, curved, and circular. In addition, the tool assembly is available in a variety of orientations in relation to a longitudinal axis of the stapling device including parallel orientations and transverse orientations.

One type of surgical stapling device includes a U-shaped end effector that has a frame having a distal transverse portion, a proximal transverse portion, and a longitudinal portion that connects the distal transverse portion to the proximal transverse portion. The proximal and distal transverse portions of the frame are spaced to define a gap. Each of the distal and proximal transverse portions has a first end coupled to the longitudinal portion and a second end spaced from the longitudinal portion. The second ends of the distal and proximal transverse portions define an opening that communicates with the gap.

Typically, in transverse type stapling devices, the cartridge assembly is supported on a clamp slide member that is initially movable between retracted and advanced positions in response to actuation of a trigger through a clamping stroke to move the stapling device between the spaced and clamped positions. Subsequently, the pusher is movable from a retracted position to an advanced position in response to actuation of the trigger through a firing stroke to eject the staples from the cartridge assembly. In addition, the knife may be displaced simultaneously with application of the staples to cut tissue.

A continuing need exists in the art for a surgical stapling device having a retractable knife assembly.

### SUMMARY

In accordance with the disclosure, a stapling device includes a handle assembly, an elongate body, a clamp slide member, and an end effector. The handle assembly includes a movable trigger and a cam member operatively coupled to the movable trigger. The cam member includes lateral walls that flexibly oppose each other and define a gap between the lateral walls. The lateral walls include respective camming protrusions extending towards each other. The elongate body has a proximal portion and a distal portion. The clamp slide member has a distal portion that defines a curved pocket and a proximal portion that defines a cam slot configured to slidably receive the camming protrusions of the cam member such that actuation of the movable trigger causes axial displacement of the clamp slide member. The end effector is supported on the distal portion of the elongate body. The end effector includes an end effector frame, an anvil assembly, and a cartridge assembly. The end effector frame has a U-shaped configuration and includes a distal transverse portion, a proximal transverse portion, and a longitudinal portion that connects the distal transverse portion to the proximal transverse portion to define a gap between the distal transverse portion and the proximal transverse portion. The distal portion of the clamp slide member is positioned within the gap and movable between retracted and advanced positions. The anvil assembly is supported on the distal transverse portion of the end effector frame. The cartridge assembly is removably received within the curved pocket of the clamp slide member.

In an aspect, the clamp slide member may include a ramp portion adjacent the cam slot.

In another aspect, the ramp portion of the clamp slide member may be configured to flex the lateral walls of the cam member and to slidably direct the camming protrusions of the cam member into the cam slot of the clamp slide member.

In yet another aspect, the proximal portion of the clamp slide member may be partially received in the gap of the cam member.

In yet another aspect, the cam member may include a cross bar interconnecting the lateral walls of the cam member.

In yet another aspect, the cam member may be formed of a polymer.

In still yet another aspect, the clamp slide member may define an elongate channel extending along a length of the clamp slide member.

In still yet another aspect, at least a portion of the clamp slide member may be received slidably within the elongate body. The clamp slide member may include lateral wings that are configured to engage the elongate body to inhibit flexing of the clamp slide member.

In still yet another aspect, the staple device may further include a thrust bar and a knife assembly. A portion of the thrust bar may be received slidably in the clamp slide member and the knife assembly may be disposed within the cartridge assembly. The thrust bar and the knife assembly may be movable as a single construct during retraction of the thrust bar.

In an aspect, the thrust bar may include an engagement head having first and second surfaces that include respective raised bumpers. The knife assembly may include opposing guides that define a slot to slidably receive the engagement head of the thrust bar.

In another aspect, the opposing guides of the knife assembly may define respective grooves to receive a corresponding raised bumper of the first or second surfaces.

In accordance with another aspect of the disclosure, a stapling device includes a handle assembly, an elongate body having a proximal portion and a distal portion, a thrust bar extending slidably through the elongate body from the handle assembly, and an end effector supported on the distal portion of the elongate body. The proximal portion of the elongate body is coupled to the handle assembly. The thrust bar includes an engagement head including first and second opposing sides having respective raised bumpers. The end effector includes an end effector frame, an anvil assembly, and a cartridge assembly. The end effector frame includes a distal transverse portion, a proximal transverse portion, and a longitudinal portion connecting the distal transverse portion to the proximal transverse portion to define a gap between the distal transverse portion and the proximal transverse portion. The anvil assembly is supported on the distal transverse portion of the end effector frame. The cartridge assembly is movable relative to the anvil assembly. The cartridge assembly includes a knife assembly. The knife assembly defines grooves that slidably receive the raised bumpers of the first and second opposing sides of the thrust bar to secure the knife assembly to the thrust bar such that the knife assembly and the thrust bar are movable as a single construct.

In an aspect, the knife assembly may include a knife holder and a knife. The knife holder may include a proximal side and a distal side. The proximal side may include first and second guides defining the grooves to slidably receive the respective raised bumpers of the first and second opposing sides of the thrust bar.

In another aspect, the distal side of the knife holder may define a slot that receives a portion of the knife to secure the knife to the knife holder.

In yet another aspect, first and second guides of the knife holder may extend towards each other from respective opposing ends of the knife holder.

In still yet another aspect, the engagement head of the thrust bar may be received slidably between the first and second guides of the knife holder.

In still yet another aspect, the staple device may further include a clamp slide member having a distal portion that defines a curved pocket. The cartridge assembly may be removably received within the curved pocket of the clamp slide member.

In an aspect, the clamp slide member may define an elongate channel along length thereof. A portion of the thrust bar may be received slidably in the elongate channel of the clamp slide member. The thrust bar may be biased towards a retracted position.

In an aspect, the handle assembly may include a cam member including lateral walls that flex relative to each other and define a gap therebetween. The lateral walls may include respective camming protrusions extending towards each other. The clamp side member may define a cam slot to receive the camming protrusions of the cam member.

In another aspect, the clamp slide member may be slidably received in the elongate body. The clamp slide member may include lateral wings that engage the elongate body to inhibit flexing of the clamp slide member.

### BRIEF DESCRIPTION OF THE DRAWINGS

Various aspects of the disclosure are described herein below with reference to the drawings, wherein:

FIG. 1 is a perspective view of a stapling device in accordance with the disclosure, illustrating a thrust bar shown in phantom;

FIG. 2 is a perspective view of the stapling device of FIG. 1, illustrating a clamp slide member shown in phantom;

FIG. 3 is a perspective view of a cam member and the clamp slide member of the stapling device shown in FIG. 2;

FIG. 4 is an enlarged view of the indicated area of detail of FIG. 3;

FIG. 5 is a perspective view of the cam member of FIG. 3;

FIG. 6 is a partial perspective view of the stapling device of FIG. 1 with a housing shown in phantom;

FIG. 7 is a cross-sectional view of the clamp slide member of FIG. 4 taken along section line 7-7 of FIG. 4;

FIG. 8 is a cross-sectional view of the clamp member of FIG. 4 taken along section 8-8 of FIG. 4;

FIGS. 9 and 10 are cross-sectional views of the clamp slide member of FIG. 7, illustrating assembly of the clamp member;

FIG. 11 is a perspective view of a thrust bar of the stapling device of FIG. 1;

FIG. 12 is a partial exploded perspective of a knife assembly and the thrust bar of FIG. 11;

FIG. 13 is a perspective view of the knife assembly of FIG. 12;

FIG. 14 is a partial perspective view of the knife assembly and the thrust bar of FIG. 13, illustrating assembly of thereof;

FIG. 15 is a cross-sectional view of the knife assembly and the thrust bar of FIG. 11;

FIG. 16 is a cross-sectional view of the knife assembly and the thrust bar of FIG. 15 taken along section line 16-16 of FIG. 15; and

FIG. 17 is an enlarged view of the indicated area of detail of FIG. 11.

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary manner, while the term “distal” is used generally to refer to that portion of the device that is farther from the clinician during use of the device in its customary manner. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

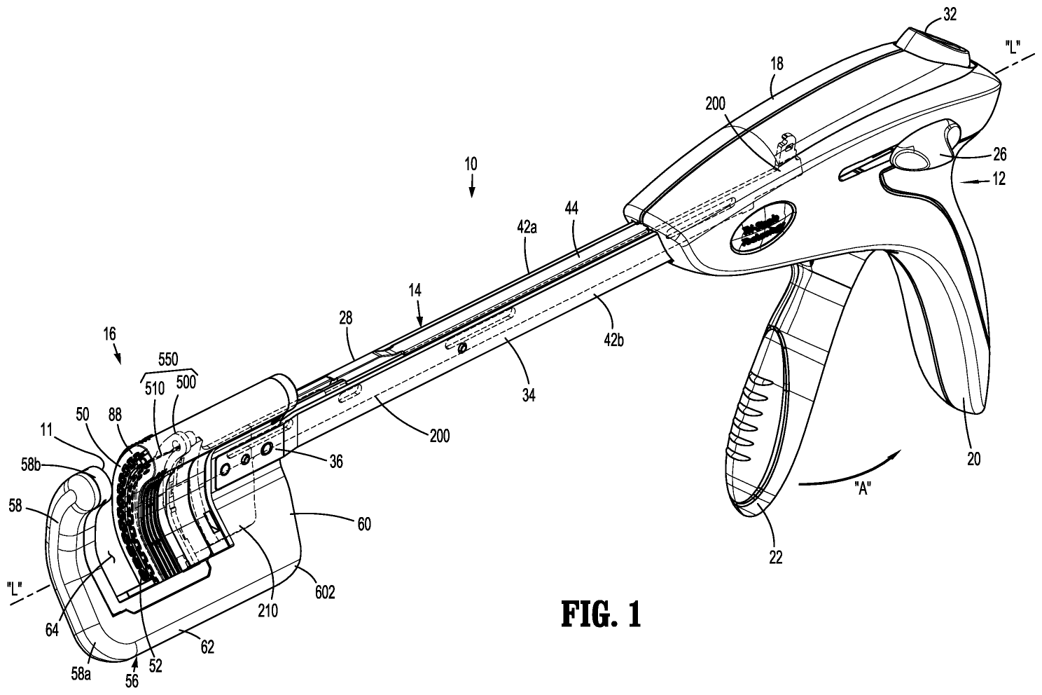

FIG. 1 illustrates the disclosed surgical stapling device shown generally as a stapling device 10. The stapling device 10 includes a handle assembly 12, an elongate body 14 that extends distally from the handle assembly 12, and an end effector 16 that is supported on a distal portion of the elongate body 14. The elongate body 14 defines a longitudinal axis “L-L”. The handle assembly 12 includes a housing 18 that defines a stationary handle 20 and supports a movable trigger 22. In an aspect, the movable trigger 22 is supported by the housing 18 to pivot in relation to the stationary handle 20 between non-actuated and actuated positions to operate the end effector 16.

The handle assembly 12 supports knobs 26 (only one shown) that are positioned on each side of the housing 18 and are movable along the housing 18 to advance and retract an alignment pin pusher 28. The alignment pin pusher 28 engages an alignment pin assembly of a cartridge assembly 50 when the cartridge assembly 50 is supported on the end effector 16. The alignment pin pusher 28 is movable via the support knobs 26 to move an alignment pin assembly between retracted and advanced positions. The handle assembly 12 also includes a release button 32 that can be depressed to move the end effector 16 from a clamped position to an unclamped position. For a more detailed description of a suitable handle assembly 12, see, e.g., U.S. Pat. No. 6,817,508 (“the '508 patent”).

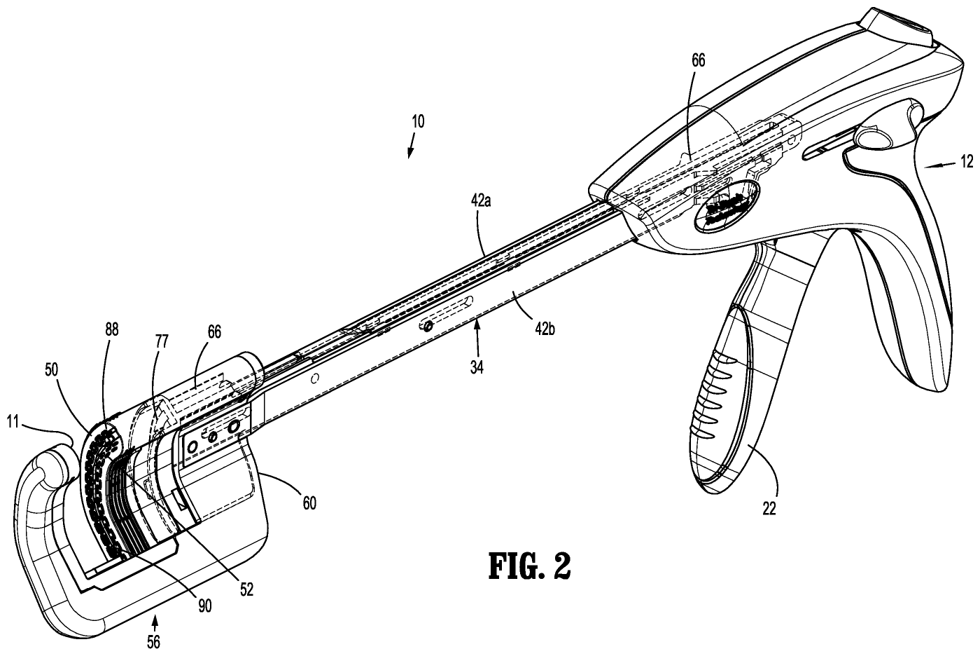

The elongate body 14 of the stapling device 10 includes a frame 34 that extends from within the handle assembly 12 to the end effector 16. The frame 34 includes a distal portion 36 that supports the end effector 16 and a proximal portion (not shown) that is supported within the housing 18 of the handle assembly 12. The frame 34 includes frame members 42a, 42b that define an elongate channel 44 that extends between the handle assembly 12 and the end effector 16. The elongate body 14 supports a clamp slide member 66 (FIG. 2), a thrust bar 200, and the alignment pin pusher 28, which are received within the elongate channel 44 defined by the frame members 42a, 42b of the stapling device 10. Each of the clamp slide member 66, the thrust bar 200, and the alignment pin pusher 28 are movable between retracted and advanced positions within the elongate channel 44 to perform the various functions of the stapling device 10, e.g., displacement of the alignment pin, displacement of the cartridge assembly 50 of the end effector 16 in relation to an anvil assembly of the end effector 16 between open and clamped positions, and displacement of a knife assembly 550.

The end effector 16 of the stapling device 10 includes an end effector frame 56 that has a U-shaped configuration and includes a distal transverse portion 58, a proximal transverse portion 60, and a longitudinal portion 62. The longitudinal portion 62 connects the distal transverse portion 58 to the proximal transverse portion 56 in spaced relation to each other to define a gap 64 that is positioned between the distal and proximal transverse portions 58, 60. In an aspect, the distal transverse portion 58 and the proximal transverse portion 60 may be curved or be formed of linear sections that are angled in relation to each other.

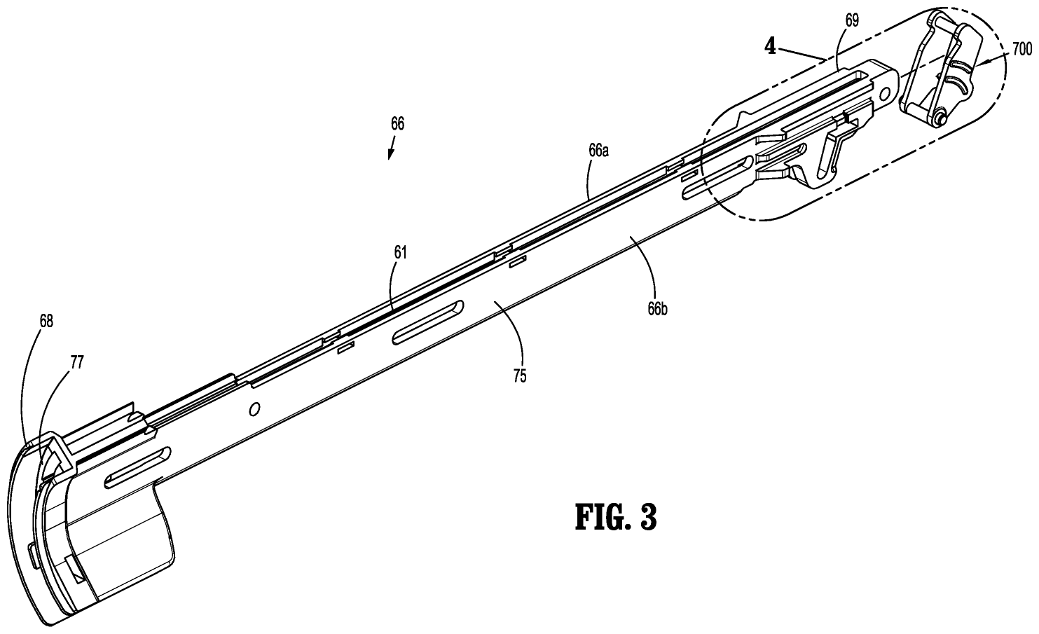

Each of the distal and proximal transverse portions 58, 60 has a first end 58a, 60a, that is coupled to (e.g., formed with) the longitudinal portion 62 of the end effector frame 56 and a second end 58b, 60b that is spaced from the longitudinal portion 62 of end effector frame 56. The second end 60b of the proximal transverse portion 60 of the end effector frame 56 is coupled to the distal portion 36 of the frame 34 of the elongate body 14 of the stapling device 10. The second end 58b of the distal transverse portion 58 of the end effector frame 56 is spaced from the longitudinal portion 62 such that the distal transverse portion 58 is supported on the longitudinal portion 62 in cantilevered fashion. The distal transverse portion 58 supports an anvil assembly 11, and the proximal transverse portion 60 defines a transverse slot that facilitates passage of the clamp slide member 66 (FIG. 3) and the thrust bar 200 through the frame 56 into the gap 64.

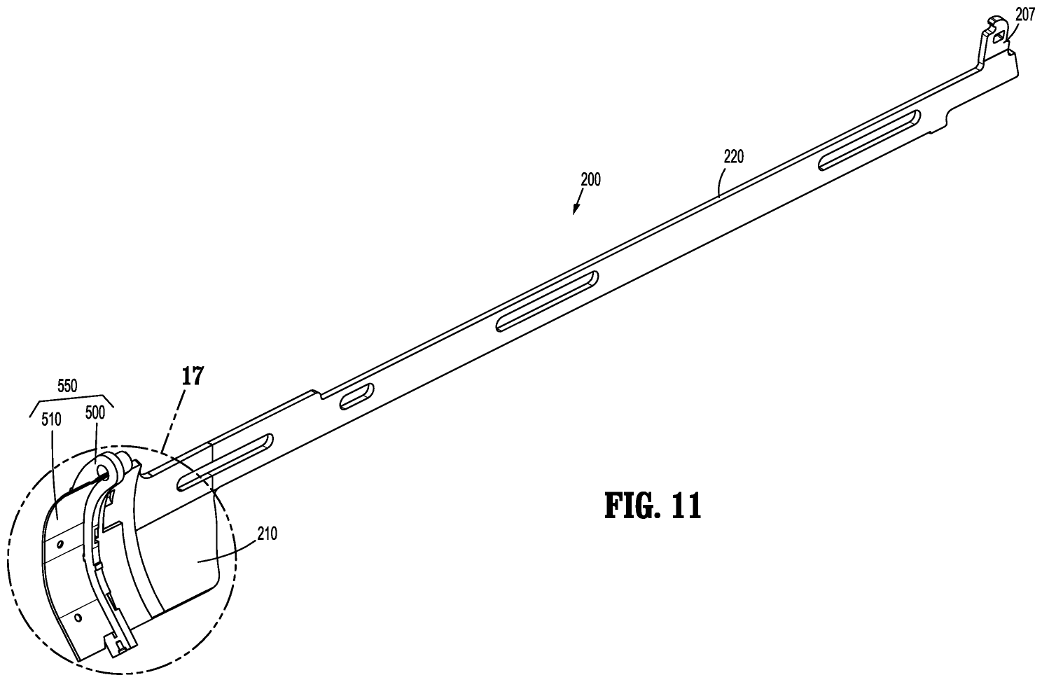

As shown in FIG. 2, the cartridge assembly 50 includes a cartridge body 57 that defines a knife slot 90 and staple receiving slots 52 that are positioned on opposite sides of the knife slot 90. The cartridge body 57 has a configuration that corresponds to the configuration of the end effector frame 56 and as such may be curved along an axis transverse to the longitudinal axis “L-L” (FIG. 1). The cartridge assembly 50 also includes a staple pusher assembly (not shown) that includes a plurality of pusher members. Each pusher member includes a plurality of fingers (not shown) configured to be slidably received within a respective staple receiving slot 52. The fingers are positioned behind staples in the staple receiving slots 52 such that advancement of fingers effects ejection of staples from the staple receiving slots 52. The cartridge assembly 50 includes a distal surface 88 that is positioned to engage and clamp tissue against the anvil assembly 11 when the cartridge assembly 50 is moved to the clamped position within the gap 64 of the end effector frame 56. The cartridge body 57 of the cartridge assembly 50 receives the knife assembly 550 (FIG. 11) and the pusher to allow movement of the knife assembly 550 and pusher between retracted and advanced positions within the cartridge body 57.

The clamp slide member 66 and the thrust bar 200 (FIG. 1) are slidably supported between frame members 42a, 42b of the frame 34 for movement between retracted and advanced positions in response to movement of the movable trigger 22 through an approximation stroke and/or a firing stroke. The clamp slide member 66 forms part of the approximation mechanism of the stapling device 10 and is movable in relation to the anvil assembly on the distal transverse portion 58 of the end effector frame 56 between retracted and advanced positions to move the cartridge assembly 50 within the gap 64 defined by the end effector frame 56 between spaced and clamped positions.

FIGS. 2 and 3 illustrate the clamp slide member 66 including a distal portion 68, a proximal portion 69, and an elongate portion 75 extending between the distal and proximal portions 68, 69. The distal portion 68 has a configuration that corresponds to the configuration of the proximal transverse portions 60 of the end effector frame 56 and defines a curved pocket 77 that is configured to releasably receive the cartridge assembly 50. The distal portion 68 is positioned within the gap 64 of the end effector frame 56.

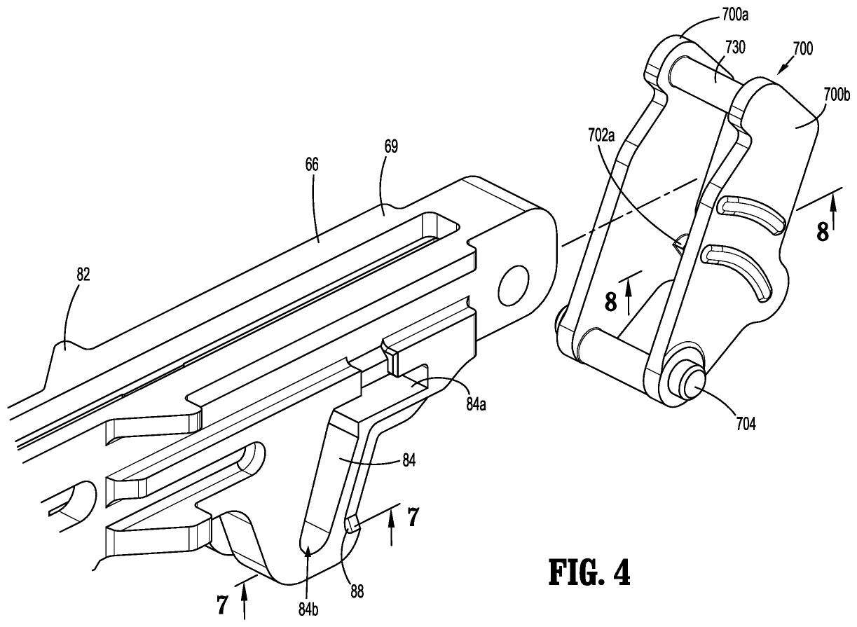

FIG. 4 illustrates the proximal portion 69 of the cam slide member 66 including lateral wings 82 that engage the frame members 42a, 42b (FIG. 1) to inhibit flexing of the cam slide member 66. In addition, the proximal portion 69 defines a cam slot 84 that receives camming protrusions 702a, 702b (FIG. 8) of a cam member 700. The cam member 700 is pivotably supported within the housing 18 (FIG. 1) of the handle assembly 12 about a pivot 704 and is operatively coupled to the movable trigger 22 (FIG. 1) of the handle assembly 12. The clamp slide member 66 includes ramp portions 88 that are disposed on opposing sides of the proximal portion 69 of the clamp slide member 66 adjacent the cam slot 84.

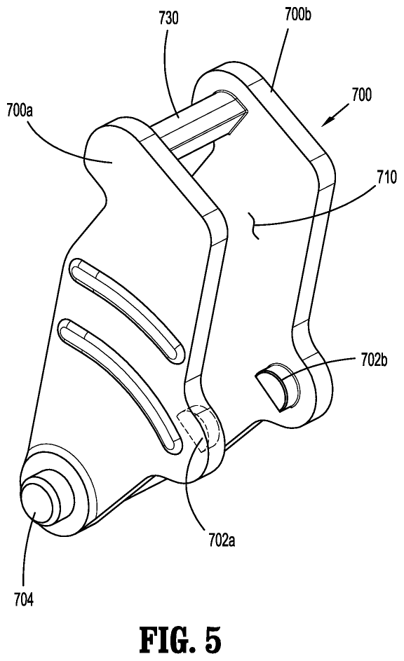

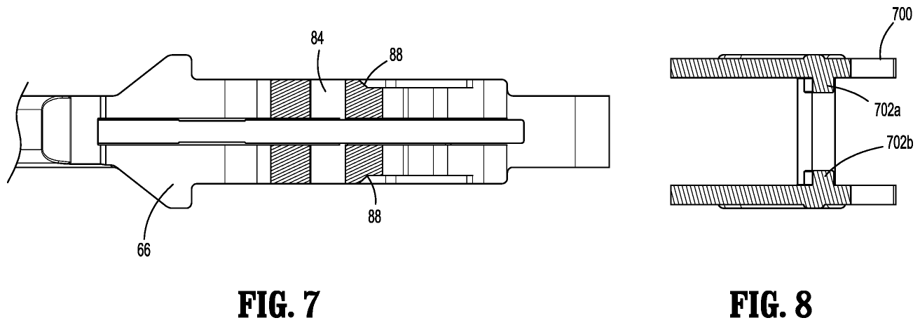

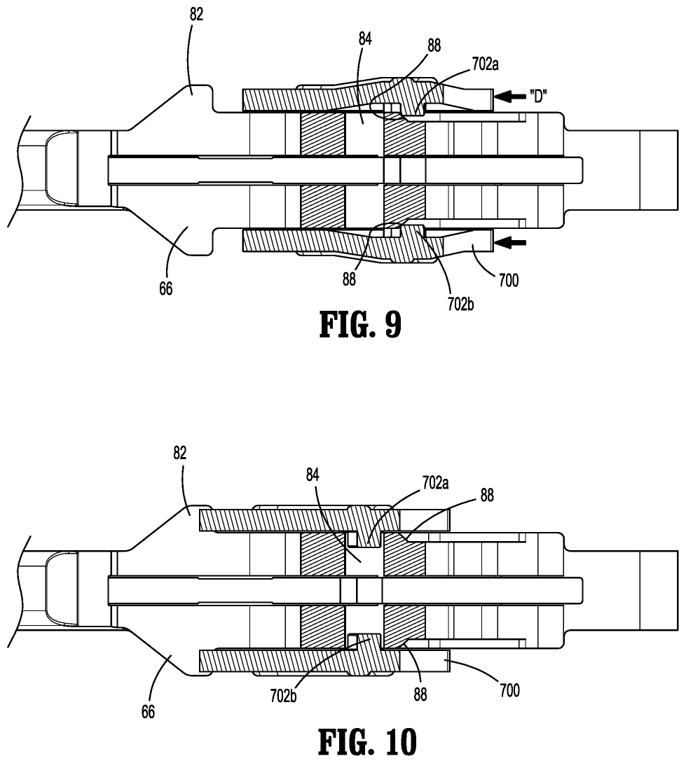

FIGS. 5-8 illustrates the cam member 700 including lateral walls 700a, 700b defining a gap 710 therebetween and a crossbar 730 that interconnects the lateral walls 700a, 700b. The lateral walls 700a, 700b oppose each other and include respective camming protrusions 702a, 702b on respective inner surfaces. In an aspect, each camming protrusion 702a, 702b may have a semi-circular shape. The camming protrusions 702a, 702b are configured to be slidably received within the cam slot 84 of the cam slide member 66 over the respective ramp portions 88 (FIG. 4). The cam slot 84 defines a proximal portion 84a (FIG. 4) that is aligned with the longitudinal axis “L-L” (FIG. 1) and a distal portion 84b that defines an acute angle with respect to the longitudinal axis “L-L”. The cam member 700 may be formed of a flexible material such as, e.g., a polymer, to enable flexing of the lateral walls 700a, 700b of the cam member 700. Under such a configuration, as shown in FIGS. 9 and 10, the lateral walls 700a, 700b flex laterally outwards when the camming protrusions 702a, 702b of the cam member 700 slide over the ramp portions 88 of the clamp slide member 66 to be received within the cam slot 84 of the clamp slide member 66. Further, manufacture of the cam member 700 using, e.g., a polymer, may reduce the cost of manufacture.

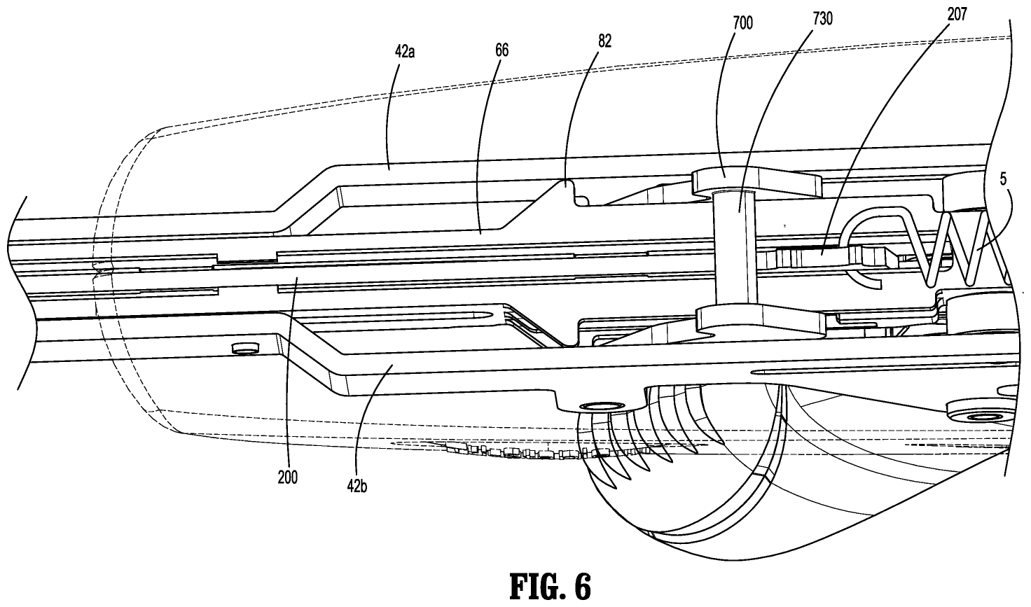

FIG. 11 illustrates the thrust bar 200 that is detachably coupled to the knife assembly 550. The knife assembly 550 includes a knife holder 500 and a knife 510 supported on the knife holder 500. The thrust bar 200 is slidably received within an elongate channel 61 (FIG. 3) defined by the clamp slide member 66. The thrust bar 200 includes an engagement head 210 and an elongate portion 220 extending proximally from the engagement head 210. The engagement head 210 is configured to engage the knife assembly 500. The proximal end portion 207 of the thrust bar 200 is coupled to a biasing member 5 (FIG. 6) supported in the housing 18 of the handle assembly 12. In this manner, the thrust bar 200 is biased to a retracted position. In aspects, the biasing member 5 may be in the form of a coil spring although the use of other biasing member types is envisioned.

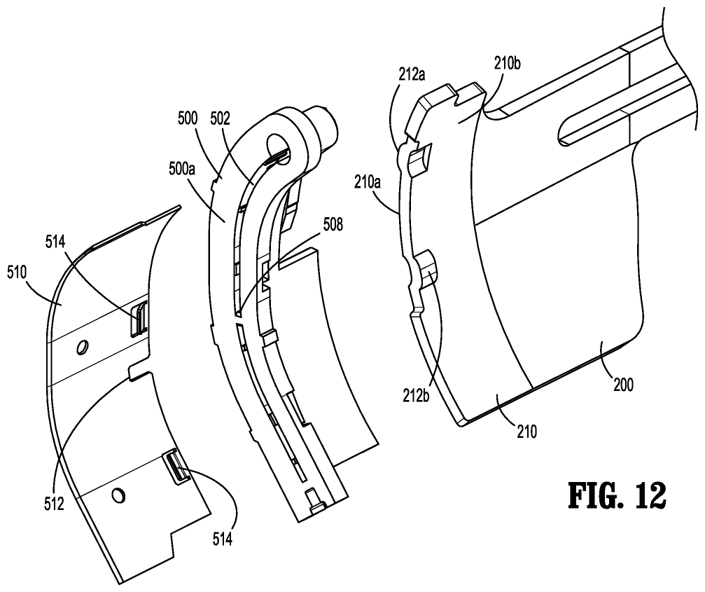

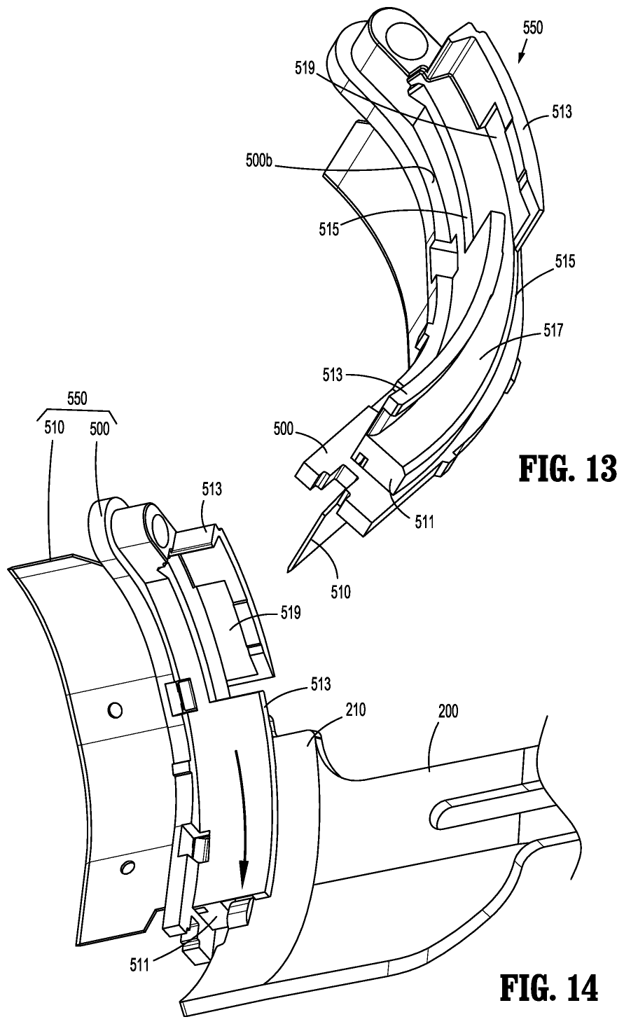

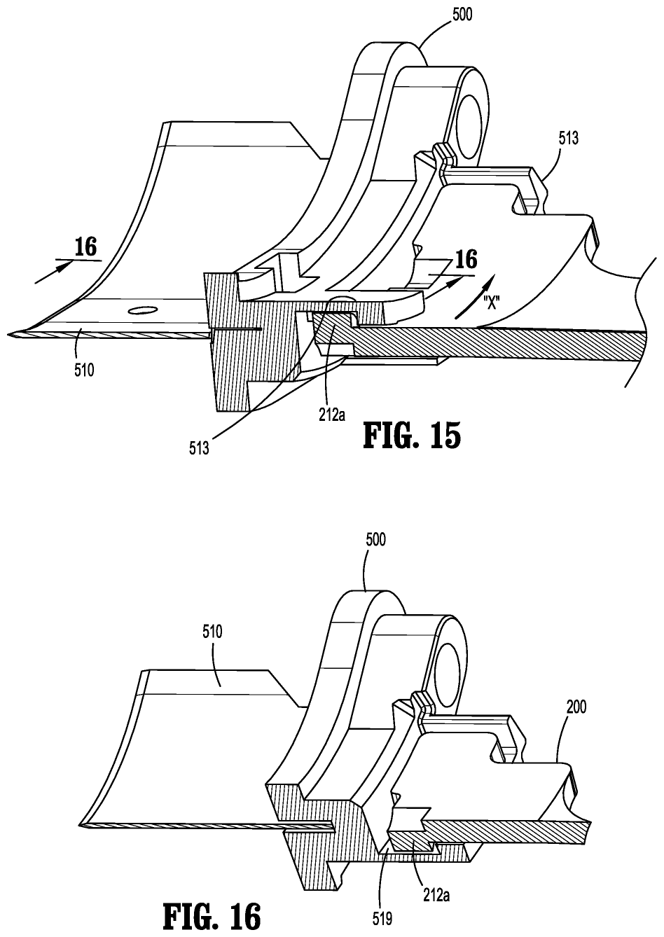

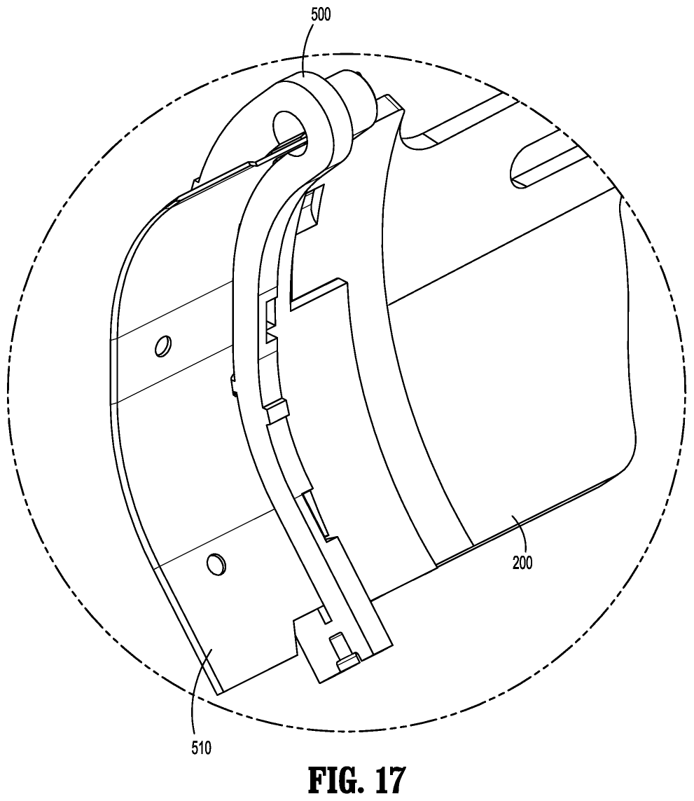

FIGS. 12 and 13 illustrates the engagement head 210 of the thrust bar 200 having a curvature corresponding to a curvature of the knife holder 500. The engagement head 210 includes first and second surfaces 210a, 210b having respective raised bumpers 212a, 212b. Each of the raised bumpers 212a, 212b has an arcuate profile. The knife holder 500 has first and second sides 500a, 500b. The first side of the knife holder 500 defines a slot 502 to receive a portion of the knife 510. The knife 510 has fingers 514 that extend laterally outwards such that when the portion of the knife 510 is received in the slot 502 of the knife holder 500, the fingers 514 of the knife 510 secure the knife 510 to the knife holder 510. Further, the knife 510 defines a cutout 512 configured to receive a cross support 508 of the knife holder 500 to facilitate alignment of the knife 510 with the slot 502 of the knife holder 500. The second side 500b of the knife holder 500 includes guides 513 that extend towards each other from respective opposing ends of the second side 500b of the knife holder 500. The guides 513 extend along opposing edges 515 of a base 511 such that the guides 513 define a slot 517 to receive a portion of the engagement head 210 of the thrust bar 200 (FIG. 14). Each guide 513 defines a groove 519 to slidably receive the corresponding raised bumper 212a, 212b of the engagement head 210 of the thrust bar 200. The engagement head 210 of the thrust bar 200 may slide through the slot 517 of the knife holder 500 in the direction of an arrow “X” (FIG. 15). In this manner, as shown in FIGS. 16 and 17, the knife 510 supported on the knife holder 500 is movable with the thrust bar 200 as a single construct, whereby the knife 510 is retractable with the thrust bar 200 as a single construct.

Initially, the cartridge assembly 50 and the anvil assembly 11 (FIG. 1) are in spaced relation, and the clamp slide member 66 (FIG. 2) and the thrust bar 200 (FIG. 1) are in the retracted position. At this point, the clinician may manually advance alignment pin pusher 28 and the alignment pin by pushing the support knob 26. The movable trigger 22 may be actuated to advance the clamp slide member 66 and the thrust bar 200 simultaneously. After clamping tissue through the approximation stroke of the stapling device 10 by advancing the clamp slide member 66, the staples may be applied to tissue and tissue may be cut through the firing stroke of the stapling device 10 by advancing the thrust bar 200. The clinician may depress the release button 32 and retract the knife 510 supported on the knife holder 500 through retraction of the thrust bar 200.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A stapling device comprising:
a handle assembly including a movable trigger and a cam member operatively coupled to the movable trigger, the cam member including lateral walls that flexibly oppose each other and define a gap between the lateral walls, the lateral walls including respective camming protrusions extending towards each other;
an elongate body having a proximal portion and a distal portion;
a clamp slide member having a distal portion that defines a curved pocket and a proximal portion that defines a cam slot configured to slidably receive the camming protrusions of the cam member such that actuation of the movable trigger causes axial displacement of the clamp slide member; and
an end effector supported on the distal portion of the elongate body, the end effector including:
an end effector frame having a U-shaped configuration and including a distal transverse portion, a proximal transverse portion, and a longitudinal portion that connects the distal transverse portion to the proximal transverse portion to define a gap between the distal transverse portion and the proximal transverse portion, the distal portion of the clamp slide member positioned within the gap and movable between retracted and advanced positions;
an anvil assembly supported on the distal transverse portion of the end effector frame; and
a cartridge assembly removably received within the curved pocket of the clamp slide member.

2. The stapling device according to claim 1, wherein the clamp slide member includes a ramp portion adjacent the cam slot.

3. The stapling device according to claim 2, wherein the ramp portion of the clamp slide member is configured to flex the lateral walls of the cam member and to slidably direct the camming protrusions of the cam member into the cam slot of the clamp slide member.

4. The stapling device according to claim 1, wherein the proximal portion of the clamp slide member is partially received in the gap of the cam member.

5. The stapling device according to claim 1, wherein the cam member includes a cross bar interconnecting the lateral walls of the cam member.

6. The stapling device according to claim 1, wherein the cam member is formed of a polymer.

7. The stapling device according to claim 1, wherein the clamp slide member defines an elongate channel extending along a length of the clamp slide member.

8. The stapling device according to claim 1, wherein at least a portion of the clamp slide member is received slidably within the elongate body, the clamp slide member including lateral wings that are configured to engage the elongate body to inhibit flexing of the clamp slide member.

9. The stapling device according to claim 1, further comprising a thrust bar and a knife assembly, a portion of the thrust bar received slidably in the clamp slide member and the knife assembly disposed within the cartridge assembly, the thrust bar and the knife assembly movable as a single construct during retraction of the thrust bar.

10. The staple device according to claim 9, wherein the thrust bar includes an engagement head having first and second surfaces that include respective raised bumpers, and the knife assembly includes opposing guides that define a slot to slidably receive the engagement head of the thrust bar.

11. The staple device according to claim 10, wherein the opposing guides of the knife assembly define respective grooves to receive a corresponding raised bumper of the first or second surfaces.

12. A stapling device comprising:
a handle assembly;
an elongate body having a proximal portion and a distal portion, the proximal portion of the elongate body coupled to the handle assembly;
a thrust bar extending slidably through the elongate body from the handle assembly, the thrust bar including an engagement head including first and second opposing sides having respective raised bumpers; and
an end effector supported on the distal portion of the elongate body, the end effector including:
an end effector frame including a distal transverse portion, a proximal transverse portion, and a longitudinal portion connecting the distal transverse portion to the proximal transverse portion to define a gap between the distal transverse portion and the proximal transverse portion;
an anvil assembly supported on the distal transverse portion of the end effector frame; and
a cartridge assembly movable relative to the anvil assembly, the cartridge assembly including a knife assembly, the knife assembly defining grooves that slidably receive the raised bumpers of the first and second opposing sides of the thrust bar to secure the knife assembly to the thrust bar such that the knife assembly and the thrust bar are movable as a single construct.

13. The staple device according to claim 12, wherein the knife assembly includes a knife holder and a knife, the knife holder including a proximal side and a distal side, the proximal side including first and second guides defining the grooves to slidably receive the respective raised bumpers of the first and second opposing sides of the thrust bar.

14. The staple device according to claim 13, wherein the distal side of the knife holder defines a slot that receives a portion of the knife to secure the knife to the knife holder.

15. The staple device according to claim 13, wherein first and second guides of the knife holder extend towards each other from respective opposing ends of the knife holder.

16. The staple device according to claim 13, wherein the engagement head of the thrust bar is received slidably between the first and second guides of the knife holder.

17. The staple device according to claim 13, further comprising a clamp slide member having a distal portion that defines a curved pocket, the cartridge assembly removably received within the curved pocket of the clamp slide member.

18. The staple device according to claim 17, wherein the clamp slide member defines an elongate channel along length thereof, a portion of the thrust bar being received slidably in the elongate channel of the clamp slide member, the thrust bar being biased towards a retracted position.

19. The staple device according to claim 18, wherein the handle assembly includes a cam member including lateral walls that flex relative to each other and define a gap therebetween, the lateral walls including respective camming protrusions extending towards each other, the clamp side member defining a cam slot to receive the camming protrusions of the cam member.

20. The staple device according to claim 19, wherein the clamp slide member is slidably received in the elongate body, the clamp slide member including lateral wings that engage the elongate body to inhibit flexing of the clamp slide member.
